(19) United States
(12) Patent Application Publication
(10) Pub. No.: US 2005/0277778 A1
(43) Pub. Date: Dec. 15, 2005

(54) Carbon dioxide production

(76) Inventors: Krishnan Viswanathan, Houston, TX (US); Solon B. Williams, Kingwood, TX (US)

Correspondence Address: LYONDELL CHEMICAL COMPANY, 3801 WEST CHESTER PIKE, NEWTOWN SQUARE, PA, 19073, US

(21) Appl. No.: 10/865,257

(22) Filed: Jun. 10, 2004

(51) Int. Cl. C07D 301/32

(52) U.S. Cl. 549/542

(57) ABSTRACT

A method for recovering a CO₂ stream free of highly reactive volatile organic compounds from an ethylene oxide production process that employs a carbonate/bicarbonate mixture to separate CO₂ and form a carbonate concentrate that is treated in a carbonate flash unit followed by a CO₂ stripper, wherein at least one of 1) an additional flash unit is employed on the carbonate concentrate upstream of the CO₂ stripper, and 2) a side draw CO₂ stream is taken from the CO₂ stripper.

### BACKGROUND OF THE INVENTION

1. Field of the Invention

This invention relates to the production of carbon dioxide (CO₂) that is essentially free, if not completely free, of highly reactive volatile organic compounds (HRVOC). More particularly, this invention relates to the production of CO₂ from an ethylene oxide (EO) production plant wherein at least a portion of the CO₂ produced is essentially free, if not totally free, of HRVOC.

2. Description of the Prior Art

For sake of clarity, this invention is described using descriptive terms for operating process units that are routinely used in a typical EO production plant.

In a conventional ethylene oxide production plant, ethylene and oxygen are reacted at an elevated temperature of from about 500° F. (F) to about 550 F under slight pressure in the presence of a catalyst to form EO. The reaction is fast, on the order of about 1 second, and high yielding, approaching 90%. The EO reaction product is normally gaseous and contains newly formed EO, unreacted ethylene, and by-products, mainly CO₂.

The EO is separated from the ethylene and by-products in a water-wash column (EO scrubber) in the manner of a solvent recovery process. The vast majority of the EO is absorbed by the water, and the ethylene and by-products are not. The resulting EO/water solution is then steam stripped and purified by fractionation (thermal distillation). The ethylene and by-products are split with the ethylene being recycled to the reactor that forms EO aforesaid, and the by-products and very minor amounts of ethylene being separately recovered for other processing and disposition. This process yields about 1.4 pounds of EO per pound of ethylene feed at high yields, e.g., about 89%. Yields can, however, vary widely from plant to plant worldwide.

EO as a liquid boils at 56 F to form a colorless gas at room temperature. EO is traded commercially as a high purity, e.g., 99.7%, technical grade chemical. Because of its volatility under normal conditions, care must be given in the storage and transportation of EO to keep it out of the ambient atmosphere. EO is an intermediary chemical useful for making a number of derivatives of commercial value. For a full and complete description of an EO production plant see U.S. Pat. No. 6,727,389 to Viswanathan.

An EO production plant is a high volume process. For example, a typical plant can produce, just as by-product, tens of thousands of pounds of CO₂ per hour that must be disposed of in an environmentally acceptable manner. This is so because the CO₂ can carry with it small amounts, parts per million (ppm) levels, of HRVOC materials such as ethylene, EO, and methane. A substantial portion of this CO₂ is sold to CO₂ vendors who process the CO₂ they purchase in an environmentally sound manner. However, such CO₂ vendors do not always purchase the full amount of CO₂ being produced at a given EO plant, thereby leaving the EO plant management with the need to dispose of the CO₂ not purchased by the CO₂ vendors. CO₂ can be vented to the ambient atmosphere, but it should not contain any HRVOC. Accordingly, it is highly desirable to be able to remove all or essentially all of the HRVOC from CO₂ produced by an EO plant and not sold to a CO₂ vendor.

This invention has non-environmental considerations as well, e.g., the employment of CO₂ in a high quality application when the environment is less of an issue.

### SUMMARY OF THE INVENTION

In accordance with this invention there is provided a method for splitting the CO₂ product of an EO plant in a manner such that the CO₂ that is not sold to a CO₂ vendor, or otherwise similarly disposed of, is processed to remove the HRVOC there from before EO plant disposition thereof.

This is accomplished by employing an additional flash unit at a strategic point in the plant process, taking a side draw stream from a special location on the existing CO₂ stripper unit in the plant, or doing both.

### BRIEF DESCRIPTION OF THE DRAWINGS

FIG. 1 shows a block diagram of the EO reactor and EO absorber and their interrelation in a conventional EO plant, including the CO₂ containing recycle stream loop that flows between the absorber and the reactor.

FIG. 2 shows a block diagram of one embodiment within this invention, and more particularly shows both the additional flash unit and the side draw embodiments of this invention.

### DETAILED DESCRIPTION OF THE INVENTION

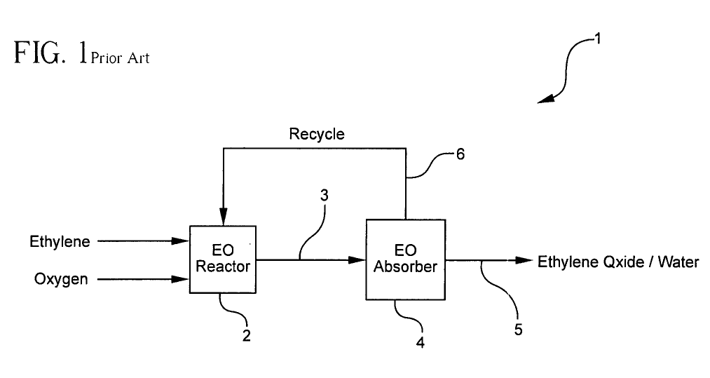

FIG. 1 shows the first two units of a typical EO production plant 1. The first unit is EO reactor 2 wherein an ethylene feed and a separate oxygen feed are reacted to form EO with the consequent formation of by-product as aforesaid. Unit 2 forms a first, normally gaseous, reaction product stream 3 that contains a major amount of newly formed EO, a substantial amount of free CO₂ (CO₂), and a minor amount of unreacted ethylene.

The second unit in FIG. 1 is the EO absorber 4 which is a water wash scrubber that operates in known manner as a solvent extractor by absorbing (dissolving) EO out of stream 3 to form a principally EO/water stream 5. Stream 5 is normally at a temperature of from about 75 to about 105 F under a pressure of about 220 psig, and is primarily a water stream that can contain from about 3 to about 5 weight percent (wt. %), based on the total weight of stream 5, EO dissolved therein. Stream 5 is then further processed in a manner that is not shown here for sake of brevity, but is shown in its entirety in U.S. Pat. No. 6,727,389. Such further processing recovers, as products of the EO plant, purified EO and/or derivatives thereof such as one or more glycols. For example, in some processes the EO is not purified, but rather is converted into ethylene glycol.

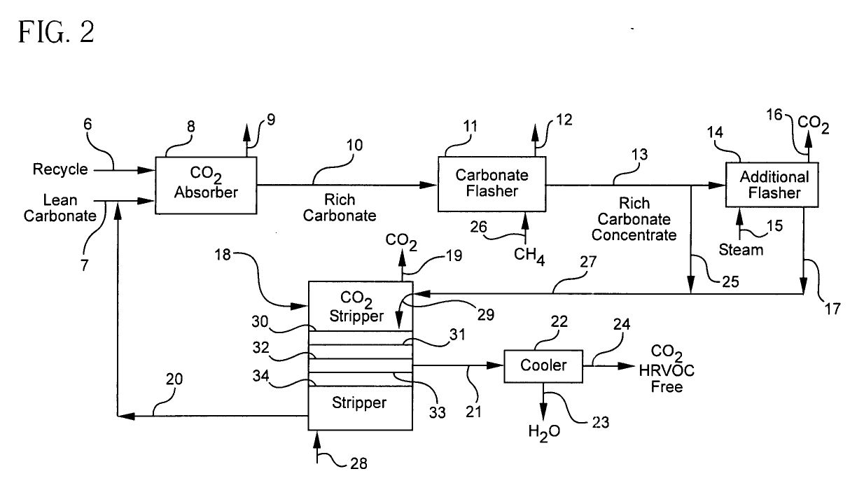

EO absorber 4 also produces a normally gaseous by-product stream 6 that is made part of the plant recycle gas loop. The recycle gas in stream 6 can contain from about 55 to about 97 wt. % methane, from about 20 to about 40 wt. % unreacted ethylene, from about 3 to about 5 wt. % CO₂, and a trace of EO, all wt. % being based on the total weight of stream 6. Stream 6 is recycled to EO reactor 2 by way of the plant recycle gas loop for reuse of the unreacted ethylene in EO reactor 2, but in so doing all or a substantial part of recycle stream 6 can be processed for the removal of CO₂ there from. This is accomplished, in one embodiment within this invention, as shown in FIG. 2.

In FIG. 2, typical EO plant recycle loop 6 is processed, using conventional EO plant terminology, in a CO₂ absorber 8, followed by a carbonate flash unit 11, and finished in a CO₂ stripper 18 to produce CO₂ product 19 that carries with it small amounts of ethylene, EO, and methane (HRVOC). This is the CO₂ product that is normally sold at least in part to CO₂ vendors. The remainder of this product that is not sold to CO₂ vendors and that contains HRVOC is what this invention addresses.

Recycle 6 is normally passed into CO₂ absorber 8, which is a packed column reactor. In absorber 8, recycle 6 is typically contacted with a mixture of alkali metal carbonates such as potassium carbonate and potassium bicarbonate. Carbonate mixture 7 contains a majority, e.g., more than about 50 wt. %, of alkali metal carbonate (carbonate), and a minority, about 50 wt. % or less of alkali metal bicarbonate (bicarbonate), both wt. % being based on the total weight of the mixture. This mixture, having less bicarbonate relative to the carbonate, is called “lean carbonate.” Lean carbonate 7 can be composed of from about 76 to about 80 wt. % water, from about 20 to about 24 wt. % of the aforesaid carbonate/bicarbonate mixture, and no carbon dioxide.

Absorber 8 is operated under conditions that favor the conversion of CO₂ to bicarbonate which conditions are, generally, a temperature of from about 195 to about 220 F under a pressure of from about 290 to about 300 psig. In absorber 8 one mole of CO₂ reacts with one mole of carbonate and one mole of water to form two moles of bicarbonate thereby removing one mole of free CO₂ from stream 6, while increasing the amount of bicarbonate present with a corresponding decrease in the amount of carbonate present. In this manner all the CO₂ present in unit 8 is eventually converted to and tied up in bicarbonate, and, thereby effectively removed from recycle 6 as free CO₂, so that the relative proportions in the carbonate/bicarbonate mixture is changed from “lean” to “rich” in unit 8. Accordingly, the rich carbonate 10 removed from CO₂ absorber 8 has more bicarbonate than carbonate, e.g., a major amount of bicarbonate and a minor amount of carbonate. Rich carbonate 10 is essentially all carbonate/bicarbonate mix but carries with it minor amounts of ethylene, EO, and methane dissolved therein.

Recycle gas 6, after processing in unit 8, is depleted in CO₂. It is then removed from unit 8 as stream 9, and returned to the recycle gas loop for return to EO reactor 2. Rich carbonate 10 is passed to carbonate flasher 11, and therein flashed with from about 5 to about 10 psig methane primarily to remove ethylene and EO from stream 10. The term “carbonate flasher” is a term of art in an EO plant in that it does not flash carbonate, but rather flashes off primarily ethylene, EO, and methane which are removed overhead from carbonate flasher 11 as stream 12. Some very slight amount of CO₂ is also flashed off, but care is taken to keep this to a minimum because flashed material stream 12 next routinely goes to residual gas absorption and compression steps, and care is taken not to load up the compressors with CO₂. Carbonate flasher 12 normally operates at a temperature of from about 190 to about 210 F at a pressure of from about 10 to about 15 psig.

A rich carbonate concentrate which is reduced, but not devoid of, its ethylene, EO, and methane content, is removed as stream 13. In a conventional EO plant, stream 13 next passes directly to CO₂ stripper 18 which is operated under conditions that favor the conversion of carbonate back to bicarbonate with the consequent release of free CO₂, two moles of bicarbonate yielding one mole of CO₂, one mole of carbonate, and one mole of water. Unit 18 operates at a temperature of from about 224 to about 230 F under a pressure of from about 5 to about 10 psig. CO₂ stripper 18 is a column that contains a large number of spaced apart trays along a substantial portion of the internal length thereof, as discussed in greater detail here in after, so that the material being treated in that column, as it progresses through the column, moves to the first tray it encounters after entering the interior of the column, and then from tray to tray toward the opposite end of the column. During such travel more and more bicarbonate is transformed into carbonate. Normally, such a column will contain at least about 16 trays and always more than seven trays.

Absent this invention, CO₂ stripper 18 normally generates an overhead CO₂ product stream 19, at about 4 to 6 psig, that is composed essentially of CO₂ and water, with ethylene, EO, and methane each present in very small (ppm) amounts, individually and collectively. Stream 19 normally is at a temperature of from about 230 to about 235 F and contains about 2 pounds of water per pound of CO₂. On a water free basis, this stream is 99.9% pure CO₂. Stream 19 is then passed to one or more coolers to condense most of the water there from and produce a pure CO₂ product that contains ethylene, EO and methane each on the ppm level, e.g., from about 200 to about 2,500 ppm collectively. This is the stream that is sold at least in part to one or more CO₂ vendors.

Bottoms 20 of CO₂ stripper 18, after flashing under an atmospheric to slight vacuum to remove the last vestiges of CO₂ present, is principally a carbonate/bicarbonate mixture which can contain more, e.g., major amount of, carbonate and less, e.g., minor amount of, bicarbonate, so that it again qualifies as lean carbonate suitable for return to and reuse in CO₂ absorber 8.

In accordance with this invention, at least one of two modifications (improvements) in apparatus and operation are employed. For sake of brevity, these are both shown in FIG. 2, however, it is to be understood that this invention does not require both modifications to be used at the same time. One or the other of such modifications can be employed by itself in an EO plant and still fall within the scope and spirit of this invention.

The first of such modifications is the use of a separate, additional steam flashing column (unit) 14. In this improvement, the rich carbonate concentrate 13 passes in whole or in part to additional flasher 14 wherein, by the use of steam 15, CO₂ is flashed off as a first separate CO₂ stream product 16 which contains HRVOC, and can be sold to a CO₂ vendor. Additional flasher 14 is operated under conditions that favor the removal of all the ethylene, EO, and methane from rich carbonate concentrate 13 along with the CO₂ in stream 16. This can leave a rich carbonate concentrate stream 17 which is at least substantially depleted in or free of HRVOC. Accordingly, if, for example, stream 17 is treated in unit 14 so as to be free of ethylene, EO, and methane, stream 17, after passage by way of line 27 to CO₂ stripper 18 and processing in unit 18, yields a second, separate CO₂ product stream 19 that is free of HRVOC and can be safely vented to the ambient atmosphere. Additional flasher 14 can be operated at a temperature of from about 190 to about 230 F under a pressure of from about −12 to about 0 psig to either severely deplete or entirely remove HRVOC there from.

The second of such modifications is the recovery of a CO₂ side draw stream 21 from CO₂ stripper 18 in addition to CO₂ stream 19. This side draw improvement can be employed alone without the use of additional stripper 14 at all, in which case stream 13 is passed directly to unit 18 by way of lines 25 and 27. This side draw can be used in combination with additional stripper 14 if stream 17 is not completely free of HVROC.

In this second improvement, side draw stream 21 is removed from the interior of stripper 18 at least “three trays down,” including the first tray to be encountered, as explained more fully here in after. Side draw stream 21, when removed in accordance with this invention, has been found to be essentially pure CO₂ and water, contain no HRVOC, and be suitable for venting. Pursuant to this invention, side draw stream 21 is drawn from below the inlet for stream 27 along the length of unit 18 at a location that is from “three to seven, inclusive, trays down,” including the first or upper most tray encountered after entry into the interior of unit 18. Side draw stream 21 can contain a substantial amount of water, and is passed through at least one cooler 22 to remove water 23 and produce a third separate CO₂ stream that is essentially pure CO₂ on a water free basis, and that is HRVOC free. Cooler 22 can be one or more fin fan cooler (not shown) that already exists in the EO plant for the purpose of cooling and dewatering stream 19.

FIG. 2 shows the first or upper 5 trays (30 through 34, inclusive) that are in the upper half of upstanding (essentially vertical) stripper 18. Tray 30 is the first tray that stream 27, upon entering the interior of unit 18, encounters as it falls downwardly inside unit 18 as shown by arrow 29. Trays 31 through 34, inclusive, are spaced apart from one another below the top (first or initial) tray 30. Tray 31, therefore is “two trays down,” i.e., the second tray stream 27 encounters after entering unit 18 or one tray below first tray 30. FIG. 2 it shows side draw 21 being taken at a location “four trays down.” This is three trays below first tray 30.

In this invention additional stripper 14 can be used alone, and without side draw 21. Or side draw 21 can be employed by itself without additional stripper 14. Or both can be used in combination as shown in FIG. 2. When additional stripper 14 is used by it self, side draw 21 not being used at all, first separate CO₂ product stream 16 can be made to contain all the HRVOC present in stream 13, while second separate CO₂ product stream 19 will be free of HRVOC. When additional stripper 14 is not used at all, and side draw 21 is used by itself, stream 19 can be made to contain all the HRVOC present in stream 13, side draw stream 21 then being HRVOC free. If additional stripper 14 does not remove, by way of line 16, all the HRVOC present in line 13, then side draw stream 21 can be used in combination with additional stripper 14 to produce an HRVOC free CO₂ stream 21. Stream 19, in such a case, could have minute quantities of HRVOC. Other combinations will be obvious to those skilled in the art.

Accordingly, if an EO plant 1 of FIG. 1 produces 50,000 pounds per hour (50 kpph) total CO₂ in stream 13 of which 30 kpph can be sold to a CO₂ vendor, 20 kpph needs to be disposed of else where. If only additional flasher 14 is employed pursuant to this invention, the plant can be operated so that stream 16 is tailored to a volume of 30 kpph which contains all the HRVOC in stream 13. This would leave stream 19 at a volume of 20 kpph and HRVOC free. If only side draw 21 is employed pursuant to this invention, the plant can be operated so that stream 19 has a volume of 30 kpph and contains all the HRVOC in stream 13, while side draw 21 has a volume of 20 kpph, all HRVOC free.

### EXAMPLE I

An EO plant as described here in above provides a recycle gas loop having a volume of about 2,000 kpph and containing about 4 wt. % CO₂ based on the total weight of the loop gas. About 1,000 kpph of this loop gas, stream 6, is split off from the recycle loop and passed to CO₂ absorber 8. Stream 9 from absorber 8 returns about 950-970 kpph of recycle gas to the loop, stream 9 having a composition consisting essentially of a mixture of ethylene and methane, up to about 1 wt. % CO₂, and a trace of EO, all wt. % being based on the total weight of stream 9.

CO₂ absorber 8 is a packed, as opposed to tray containing, column that operates at a temperature of about 200 F under a pressure of about 290 psig, and contacts about 2,000 kpph of lean carbonate 7 with the incoming recycle stream 6 to convert all the CO₂ in stream 6 to bicarbonate in the manner aforesaid. Lean carbonate stream 7 consists essentially of about 22 wt. % of a mixture of potassium carbonate and potassium bicarbonate (carbonate/bicarbonate), about 78 wt. % water, and no CO₂, all wt. % being based on the total weight of stream 7.

Rich carbonate stream 10 is formed in absorber 8 and removed there from to carbonate flasher 11 from which stream 12 in the volume of about 3 kpph is removed, stream 12 consisting essentially of ethylene, EO, methane, steam, and a slight amount of CO₂. Stream 12 is sent to residual gas absorbing and compressing operations.

Carbonate flasher 11 is operated at about 200 F under a pressure of about 15 psig using 1 kpph of methane at about 10 psig as the stripping medium. This yields a rich carbonate concentrate stream 13 at about 190 F which consists essentially of carbonate/bicarbonate mixture having a major amount of potassium bicarbonate, no CO₂, and about 50 ppm of a mixture of ethylene, EO, and methane.

Rich carbonate concentrate 13 is passed to additional flasher 14 which uses steam as the stripping medium, and operates at a temperature of about 220-230 F under a pressure of about −10 psig. In additional flasher 14 a first separate CO₂ product stream 16 is formed which is recovered as overhead in the volume of about 5-10 kpph. Stream 16 contains all the ethylene, EO, and methane that were present in stream 13. The bottoms product 17 of flasher 14 consists essentially of a rich carbonate/bicarbonate mixture which is passed to CO₂ stripper 18 by way of line 27.

CO₂ stripper 18 contains 16 spaced apart trays, and is operated at a temperature of about 225 F under a pressure of about 5 psig using steam 28 as the stripping medium. In stripper 18 a majority of the potassium bicarbonate therein is converted to potassium carbonate thereby freeing CO₂. The ethylene, EO, and methane were removed by way of first, separate CO₂ stream 16. Accordingly, the second, separate CO₂ stream 19 is removed from stripper 18 in the volume of about 20-30 kpph, is HRVOC free, and is ready for disposition other than sale to a CO₂ vendor, e.g., venting. Stream 19 can then be cooled in one or more fin fan coolers to remove water there from.

The carbonate/bicarbonate mixture in stripper 18, after processing as aforesaid in stripper 18, is recovered as bottoms 20 there from. Stream 20, after a separate vacuum steam stripping operation to remove the last vestiges of CO₂ there from, contains no CO₂. After such processing stream 20 contains a carbonate/bicarbonate mixture whose potassium carbonate content is significantly greater than its potassium bicarbonate content so that it is suitable for use as lean carbonate. It, therefore, is returned by way of line 20 for reuse in absorber 8.

### EXAMPLE II

The process of Example I above was carried out except that additional flasher 14 was not employed. Instead, stream 13 was passed directly to CO₂ stripper 18 by way of lines 25 and 27, and a side draw stream 21 was removed from stripper 18 separate from, and in addition to, CO₂ product stream 19.

In this example, side draw stream 21 was removed from stripper 18 one tray below first tray 30 that entering stream 27 initially encountered inside stripper 18, i.e., “two trays down” after stream 27 enters stripper 18. This “two trays down” location is shown as tray 31 in FIG. 2. In this example side draw stream 21 contained no EO or methane, but its ethylene content was only 90 wt. % less than that of stream 27, second, separate CO₂ product stream 19 containing the remainder of the ethylene, EO, and methane that was present in stream 13. Side draw stream 21 was sent to fin fan coolers that already existed in the plant to remove water there from and produced a third separate CO₂ product that was 99.9 wt. % CO₂ on a water free basis.

### EXAMPLE III

The process of Example II above was repeated except that side draw stream 21 was taken “four trays down,” i.e., from tray 33, as shown in FIG. 2. This was four trays down after stream 27 entered unit 18, and 3 trays below upper most tray 30 which stream 21 encountered first. The side draw stream of this Example contained no ethylene, EO, or methane.

1. In a method for removing at least one CO₂ stream that is essentially free of HRVOC from a process for forming EO which process employs an ethylene and oxygen reaction step to form a first reaction product that contains EO, water, CO₂, and ethylene from which a mixture of EO and water is separated thereby leaving a separate recycle stream that contains CO₂, ethylene, and EO, said recycle stream being treated in a CO₂ absorber with a mixture of carbonate/bicarbonate to convert CO₂ to bicarbonate and thereby form a rich carbonate stream, said rich carbonate stream is treated in a carbonate flasher that employs methane to remove most of the ethylene and EO there from and produce a separate rich carbonate concentrate, said rich carbonate concentrate being treated in a CO₂ stripper to convert bicarbonate to carbonate and free CO₂, said CO₂ stripper being a trayed column that contains more than seven trays therein so that as said rich carbonate concentrate moves from the first tray it encounters upon entering said CO₂ stripper to subsequent trays in said stripper more and more bicarbonate is converted to carbonate, the improvement comprising, at least one of 1) employing a separate and additional flasher column between said carbonate flasher and said CO₂ stripper, said additional flasher being operated under conditions which favor the conversion bicarbonate to carbonate and thereby free CO₂ there from and to remove from said additional flasher a first separate CO₂ stream containing ethylene, EO, and methane thereby producing for treatment in said CO₂ stripper a rich carbonate concentrate that is reduced in if not essentially free of ethylene, EO, and methane content, and 2) removing from said CO₂ stripper a separate CO₂ side draw stream at least 3 trays down in said CO₂ stripper, said side draw stream being free of ethylene, EO, and methane.

2. The method of claim 1 wherein said side draw stream is not removed from said CO₂ stripper and only said additional flasher column is employed whereby said first separate CO₂ stream contains the ethylene, EO, and methane that is present in said rich carbonate concentrate, and said rich carbonate concentrate passing from said additional flasher column to said CO₂ stripper is free of ethylene, EO, and methane, whereby a second separate CO₂ stream is recovered from said CO₂ stripper which is free of ethylene, EO, and methane.

3. The method of claim 1 wherein said additional stripper is not employed and only said side draw stream from said CO₂ stripper employed, whereby CO₂ is recovered from said CO₂ stripper as said second separate CO₂ stream which contains the ethylene, EO, and methane that is present in said rich carbonate concentrate, and said separate CO₂ side draw stream is recovered which is free of ethylene, EO, and methane.

4. The method of claim 1 wherein said carbonate/bicarbonate mixture is composed of potassium carbonate and potassium bicarbonate.

5. The method of claim 4 wherein said recycle stream is treated with said carbonate/bicarbonate mixture in said CO₂ absorber to convert all the CO₂ in said recycle stream to potassium bicarbonate and form said rich carbonate stream containing essentially a mixture of potassium carbonate and potassium bicarbonate which is rich in bicarbonate and contains essentially no CO₂ but contains minor amounts of unreacted ethylene, EO, and methane, said rich carbonate stream is subjected to flashing conditions in said carbonate flasher which removes most of the unreacted ethylene, EO, and methane from same thereby leaving a rich carbonate concentrate that is concentrated in a mixture of potassium carbonate and potassium bicarbonate but still contains minor amounts of unreacted ethylene, EO, and methane, said rich carbonate concentrate being subjected to stripping conditions in said CO₂ stripper column wherein potassium bicarbonate is converted to potassium carbonate thereby freeing CO₂ and producing a lean carbonate stream that is recycled to said CO₂ absorber.

6. The method of claim 1 wherein said side draw stream is taken from a location on said CO₂ stripper that is from 3 to 7, inclusive, trays down from the top of said CO₂ absorber where said rich carbonate concentrate enters the interior of said CO₂ absorber.
